(19) United States
(12) Patent Application Publication
(10) Pub. No.: US 2009/0280472 A1
(43) Pub. Date: Nov. 12, 2009

(54) Method for Detection of Antigens

(75) Inventors: Sulatha Dwarakanath, Austin, TX (US); John G. Bruno, San Antonio, TX (US); Poornima M. Rao, Austin, TX (US)

Correspondence Address: FISH & RICHARDSON P.C., P.O BOX 1022, Minneapolis, MN, 55440-1022, US

(73) Assignee: NANO SCIENCE DIAGNOSTICS, INC., Austin, TX (US)

(21) Appl. No.: 11/910,598

(22) PCT Filed: Nov. 30, 2006
(86) PCT No.: PCT/US06/61382

§ 371 Date: Oct. 3, 2007

Related U.S. Application Data
(60) Provisional application No. 60/741,349, filed on Nov. 30, 2005.

(51) Int. Cl. C12Q 1/70; G01N 21/75; G01N 33/00; C12Q 1/02; G01N 33/569; G01N 33/53

(52) U.S. Cl. 435/5; 436/164; 436/86; 435/29; 435/7.32; 435/7.1

(57) ABSTRACT

The field of the invention relates generally to the detection of antigens, including, but not limited to, quantum dots (Qdots) and metal oxide nanoparticles. More specifically, the invention relates to the detection of antigens on a surface or in a source, which antigens include bacteria, viruses, and small proteins. In some embodiments, the invention can be used to detect biological warfare agents, such as anthrax and ricin. In some embodiments, the invention can be used for early detection of diseases in human and animals. The invention may utilize a swab-test and may further utilize a filtration process, such as with a syringe-disc.

### CROSS REFERENCE

This application claims priority to and benefit of U.S. Provisional Application Ser. No. 60/741,349, filed on Nov. 30, 2005.

### RELATED PATENT APPLICATION

The following co-pending and co-assigned applications contain related information and are incorporated herein by reference: (1) U.S. patent application Ser. No. 11/292,604, filed Dec. 2, 2005, entitled “Method and Apparatus for Low Quantity Detection of Bioparticles in Small Sample Volumes” having Srinagesh Satyanarayana and Sulatha Dwarakanath as inventors; and (2) U.S. patent application Ser. No. 11/222,093, filed Sep. 8, 2005, entitled “Method for Detection and Decontamination of Antigens by Nanoparticle-Raman Spectroscopy” having Sulatha Dwaraknath and John G. Bruno as inventors.

### STATEMENT OF FEDERALLY SPONSORED RESEARCH/DEVELOPMENT

The present invention was made in connection with research pursuant to Department of Defense Contract No. W9132T-040C-0030.

### FIELD OF THE INVENTION

The field of the invention relates generally to the detection of antigens, including, but not limited to, quantum dots (Qdots) and metal oxide nanoparticles. More specifically, the invention relates to the detection of antigens on a surface or in a source, which antigens include bacteria, viruses, and small proteins. In some embodiments, the invention can be used to detect biological warfare agents, such as anthrax and ricin. In some embodiments, the invention can be used for early detection of diseases in human and animals. The invention may utilize a swab-test and may further utilize a filtration process, such as with a syringe-disc.

### BACKGROUND OF THE INVENTION

The flurry of anthrax mailings and the contamination of Senator Thomas Daschle's Capitol Hill headquarters complex in late 2001, which later cost an estimated $41.7 million to decontaminate by chlorine dioxide, underscore the need for detection and neutralization technologies to combat civilian bioterrorism and military biowarfare attacks. Development of method that could both aid in detection and decontamination of biological warfare agents (also referred to as biowarfare agents) in building interiors would be a valuable asset in homeland defense and a useful military tool.

Therefore, there is a need to develop a method for the detection of biowarfare agents. Such technology would also be useful in hospitals, surgical suites, industrial clean rooms. There is further a need for such an apparatus and method to afford sensitive detection in a single process, and which utilizes devices that are generally available. Such technologies could promote rapid and much more cost effective detection and decontamination of biological warfare agents in public facilities contaminated by the actions of bioterrorists. Such technologies would also be useful in early detection of diseases in human and animals. And, a rapid, sensitive, and easy to use technology would also be beneficial for point of Care Testing (POCT) in hospitals and doctors offices.

There is further a need that the target antigen may be detected at very low amounts, such as at concentrations as low as about 10 cfu/ml and 4 μg/l.

Quantum dots are particles of matter so small that the addition or removal of an electron changes their properties. Quantum dots (QDs) have high fluorescence efficiency, lack photobleaching, and have long fluorescence (decay) lifetimes [H. Harma, T. Soukka, T. Lovgren, “Europium nanoparticles and time-resolved fluorescence for ultrasensitive detection of prostate-specific antigen,” Clin. Chem. 47 (2001) 561-568; T. Soukka, J. Paukkunen, H. Harma, S. Lonnberg, H. Lindroos, T. Lovgren, “Supersensitive time-resolved immunofluorometric assay of free prostate-specific antigen with nanoparticle label technology,” Clin. Chem. 47 (2001) 1269-1278]. These properties allow QDs to be ultrasensitive and therefore compete with conventional fluorescent dyes for many applications.

A composition and method has been discovered [co-pending and co-assigned U.S. patent application Ser. No. 11/222,093, filed Sep. 8, 2005] for detection and decontamination of antigens by nanoparticle-Raman spectroscopy, which comprises a fluorescent nanoparticle conjugated to a substance capable of binding specifically to an antigen and exposing the location containing the fluorescent nanoparticle and antigen to a wavelength of light capable of exciting the fluorescent nanoparticle. For instance, in an embodiment disclosed therein, a method was described of detecting an antigen comprising: (a) obtaining a fluorescent nanoparticle conjugated to a substance capable of binding specifically to an antigen to form a conjugated fluorescent nanoparticle; (b) placing the conjugated fluorescent nanoparticle in a location where the antigen is suspected to be; (c) exposing the location to a wavelength of light capable of exciting the conjugated fluorescent nanoparticle; (d) measuring fluorescence emission of the conjugated fluorescent nanoparticle; and (e) observing the wavelength of the measured fluorescence emission of step (d) in comparison with the wavelength of the fluorescence emission of the conjugated fluorescent nanoparticles that have not been exposed to the antigen wherein the conjugated fluorescent nanoparticle exhibits a lower emission wavelength upon binding to the antigen.

Furthermore, a method and apparatus was discovered [co-pending and co-assigned U.S. patent application Ser. No. 11/292,604, filed Dec. 2, 2005] for low quantity detection of bioparticles in small sample volumes (i.e., nanoliter/picoliter quantities of a sample). The apparatus involved a very small and low cost apparatus that contains a fluorometer. The detection process used the fluorescence of nanoparticles. Dielectrophoresis can used to concentrate, mix and position the target particles with regard to the light sensor such that maximum detection efficiency could be achieved.

### BRIEF SUMMARY OF THE INVENTION

The invention relates to antibody-nanoparticle (NP) or other receptor-NP conjugates for detection of antigens, such as for detection of antigens used as biological warfare agents (like anthrax or ricin) and early detection of diseases in human and animals. An apparatus and method has been discovered utilizing receptor (antibody or aptamer)-conjugated nanoparticles (NPs) or quantum dots (QD) capable of fluorescence scanner-based detection of antigens. Such apparatus and can be in, for example, a swab-base swipe test (i.e., swabbing of antibody-NP conjugates onto building interior surfaces). In embodiments of the invention, agent fluorescent NP-based immunoassay test kits can be used with any general fluorometer used for detection purposes. The invention may be used to detect antigens having concentrations less than 10,000 cfu/ml and may be used to detect the presence of very low concentrations of antigens (even small proteins), such as at concentrations of antigens as low as about 10 cfu/ml and 4 μg/ml.

In an embodiment of the invention, the present invention is a method of detecting an antigen comprising: (a) obtaining a sample from a surface or other source where an antigen is suspected to be (such as by using a swab-test); (b) obtaining a fluorescent nanoparticle conjugated to a substance capable of binding specifically to the antigen; (c) interacting the sample with the fluorescent nanoparticle such that antigen, if present, is bound to the conjugated fluorescent nanoparticles; (d) exposing the conjugated fluorescent nanoparticle of the resulting material to a wavelength of light capable of exciting the conjugated fluorescent nanoparticle; (e) measuring fluorescence emission of the conjugated fluorescent nanoparticle; and (f) observing the wavelength of the measured fluorescence emission of step (e) in comparison with the wavelength of the fluorescence emission of the conjugated fluorescent nanoparticles that have not been exposed to the antigen wherein the conjugated fluorescent nanoparticle exhibits a lower emission wavelength upon binding to the antigen.

Such interacting step may further comprise incubating the sample with the fluorescent nanoparticle.

Embodiments of the invention may further comprise a filtration method (such as a syringe-disc) that may be utilized in embodiments of the present invention. In this embodiment, after interacting the sample with the fluorescent nanoparticle, the resulting material may be passed through a filtering material (such as a disc), that has a pore size selected dependant upon the size of the antigen, to filter out the unbounded conjugated fluorescent nanoparticles from this interacted material. The nanoparticles that do not pass through the disc may them be exposed, measured, and observed as described above. This testing can be done so on, for example, the filter disc itself, by reverse flushing of the disc (to get the bounded conjugated fluorescent nanoparticles out of the filter),or by washing the conjugated fluorescent nanoparticles out in a buffer (such as a Phosphate Buffered Saline (PBS) buffer).

By such embodiments, the emission peaks can be read off and measured quantitatively.

Another embodiment of the present invention is a method for detecting two or more types of antigen comprising a first and second fluorescent nanoparticle conjugated to substances capable of binding specifically to the two or more types of antigen to form a first and second conjugated fluorescent nanoparticles wherein the first and second conjugated nanoparticles emit at different wavelengths and exhibit a lower emission peak wavelength upon binding to the two or more types of antigen.

The foregoing has outlined rather broadly the features and technical advantages of a number of embodiments of the present invention in order that the detailed description of the present invention that follows may be better understood. Additional features and advantages of the invention will be described hereinafter.

### BRIEF DESCRIPTION OF THE DRAWINGS

The foregoing summary as well as the following detailed description of the preferred s embodiment of the invention will be better understood when read in conjunction with the appended drawings. It should be understood, however, that the invention is not limited to the precise arrangements and instrumentalities shown herein. The components in the drawings are not necessarily to scale, emphasis instead being placed upon clearly illustrating the principles of the present invention. Moreover, in the drawings, like reference numerals designate corresponding parts throughout the several views.

The invention may take physical form in certain parts and arrangement of parts. For a more complete understanding of the present invention, and the advantages thereof, reference is now made to the following descriptions taken in conjunction with the accompanying drawings, in which:

FIG. 1 is a diagram of Nano-Ab-Tag that can be used in an embodiment of the present invention.

FIG. 2 is a flow diagram illustrating an embodiment of the present invention.

FIG. 3 is a flow diagram illustrating steps that may be utilized in an embodiment of the present invention.

FIG. 4 is an illustration reflecting steps that may be utilized in an embodiment of the present invention.

FIG. 5 is a graph reflecting the fluorescent output measured for various concentrations of Male Specific Coliphage (“MS2”) (viral stimulant) utilized in embodiments of the invention.

FIG. 6 is a graph reflecting the fluorescent output measured for these various concentrations of Ovalbumin (“OV”) (Ricin stimulant) utilized in embodiments of the invention.

FIG. 7 is a graph reflecting the fluorescent output measured for these various concentrations of Erwinia herbicola (“EH”) (Yersinia pestis stimulant) utilized in embodiments of the invention.

FIG. 8 is a graph reflecting the fluorescent output measured for these various concentrations of Bacillus Globigii (“BG”) (anthrax stimulant) utilized in embodiments of the invention.

FIG. 9 is a graph reflecting the unfiltered spectra received for a mixture of (a) dead Listeria monocytogenes bound to Ab-Lake Placid Blue QDs (498 nm), (b) live E. coli O111:B4 bound to Ab-Adirondack Green QDs (522 nm), (c) dead Campylobacter bound to Ab-Birch Yellow QDs (588 nm) and (d) dead E. Coli O157:h7 bound to Ab-Fort Orange QDs (605 mm).

FIG. 10 is a graph illustrating the specta received from dead Listeria monocytogenes bound to Ab-Lake Placid Blue QDs (498 nm).

FIG. 11 is a graph illustrating the specta received from live E. coli O111:B4 bound is to Ab-Adirondack Green QDs (522 nm).

FIG. 12 is a graph illustrating the specta received from dead Campylobacter bound to Ab-Bircb Yellow QDs (588 nm).

FIG. 13 is a graph reflecting detection of E. Coli O157:h7 bacteria in a food sample using an embodiment of the invention.

FIG. 14 is a graph reflecting detection of Salmonella typhimurium bacteria in a food sample using an embodiment of the invention.

FIG. 15 is a graph reflecting detection of OV in a serum sample using an embodiment of the invention.

### DEFINITIONS

An “antibody” is an immunoglobulin molecule that only interacts with the antigen that induced its synthesis in cells of the lymphoid series, or with an antigen closely related to it.

An “antigen” is a substance capable of inducing synthesis of an antibody and being bound by such antibody. This substance is selected from the group including but not limited to bacteria, virus, viral particles and protein.

“Aptamers” are specific RNA or DNA oligonucleotides or proteins which can adopt various three dimensional configurations. Because of this aptamers can be produced to bind tightly to a specific molecular target.

“Bacteria” are one cell organisms.

“CFU” are colony forming units.

“Fluorescence” is the emission of light of one wavelength upon absorption of light of another wavelength.

“Quantum dots” or “QDs” are particles of matter so small that the addition or removal of an electron changes their properties.

“Raman Emission Peak” is the peak at about 460 nm wavelength for water.

“Wavelength” is the distance between two waves of energy.

### DETAILED DESCRIPTION OF THE INVENTION

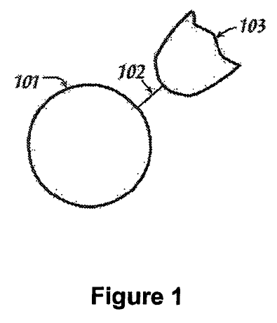

As disclosed in co-pending and co-assigned U.S. patent application Ser. No. 11/222,093, filed Sep. 8, 2005, NPs (sometimes termed as semiconductor NPs), can be used to sensitively detect antigens, including, but not limited to, bacteria, virus, and proteins. Such NPs, which can be composed of CdSe/ZnS quantum dots (QDs) exhibit change in the Raman Emission Peak when conjugated to antibodies or DNA aptamers that are bound to bacteria or other antigens. Such a Nano-Ab-Tag can be formed, as shown in FIG. 1. The intensity of the Raman Emission Peak was found to increase with the number of bound antigen, which was a very minor component of the natural fluorescence spectrum of these QDs. The NPs can be conjugated to specific antibodies and used to sensitively detect specific antigens by both fluorescence microscopy and spectrofluorometry. A fluorescence surface scanner can be used without the need for wash steps to eliminate background fluorescence because the emission peak for the unbound NPs is at a different wavelength. A variation can be to use quantum confined nanosize particles that fluoresce and can be conjugated to an antibody or nucleic acid. For instance, nanoparticles, either semiconductor or metal oxide with a lanthanide core, can be conjugated to an antibody or nucleic acid, through a chemical linkage.

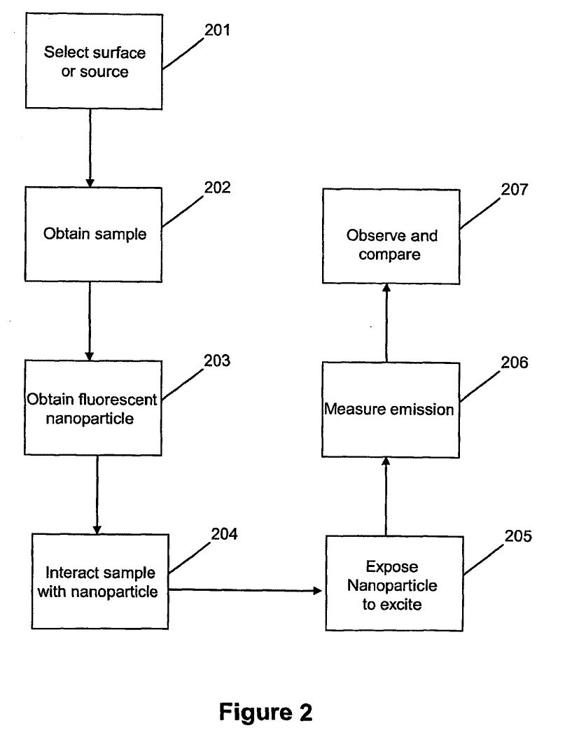

Referring to FIG. 2, which illustrates an embodiment of the invention, in step 201, a surface, such as a wall, floor, building interior, etc., or other source is selected for investigation to determine whether a suspected antigen is present. The surface or source may also be biological, such as from a human or animal. For example, the sample would be obtained from a body fluid, such as blood, urine, stool sample, saliva, or spinal fluid.

In step 202, a sample is obtained from that surface or source, such as by swiping the surface or source with a material that will obtain, but not effect or alter, the suspected antigen. One manner in which this can be done is by a swab-test (also referred to as a swab-base swipe test), such as the following. An area that is selected to be tested is swabbed (such as with a wet swab). The swab can then dipped into a release buffer for a period of time, generally at least two minutes, and more typically at least five minutes, to yield a sample (which will include the suspected antigen, if present on the surface or source). The swab can then be disposed of and the sample can be used for testing. In another embodiment, antigens on the surface can be obtained by washing off the surface with a liquid spray and collecting the liquid.

In step 203, a fluorescent nanoparticle is obtained that has conjugated to it a substance capable of binding specifically to the suspected antigen to form a conjugated fluorescent nanoparticle. Such fluorescent nanoparticle may be QDs, such as those made by Quantum Dot Corp. (now Invitrogen Corp., Carlsbad, Calif.), like Qdot 655 nm. As illustrated in FIG. 1, the fluorescent nanoparticle 101 is bound to the antibody 103 through molecular bridge 102. The antibody 103 is selected such that it is capable of binding to the surface of the suspected antigen.

In step 204, the sample obtained in step 202 is interacted with the fluorescent nanoparticle to form a resulting material. If the suspected antigen is present within the sample, the antigen will bind with the conjugated fluorescent nanoparticles as anticipated and the resulting material will comprise bounded conjugated fluorescent nanoparticles. If the suspected antigen is not present within the sample, the conjugated fluorescent nanoparticles will not have any antigen bound to it. In one embodiment of the invention, step 204 includes incubating the sample with the fluorescent nanoparticle. For instance, 1 ml of the required sample can be incubated with about 10 μl (5 μg) of the conjugated fluorescent nanoparticles for at least around 10 minutes, and more particularly at least around 15 minutes.

In step 205, the resulting material is exposed to a wavelength of light capable of exciting the conjugated fluorescent nanoparticle. In step 206, the fluorescence emission of the resulting materials is measured, including, in particular, the emission of the conjugated fluorescent nanoparticle, if any, present in the interacted material. Such exposure and measurement can be performed on any general purpose fluorometer that can read emission from 300 to 700 nm, such as, for example, the Cary Eclipse Fluorometer from Varian, Inc., (Walnut Creek, Calif.) which scans fluorescence emissions from 200-850 nm with picomolar sensitivity or the Picofluor from Turner Biosystems, Inc. (Sunnyvale, Calif.), which is an off-the-shelf handheld or portable fluorometer. Steps 205-206 can be completed in a variety of time frames, including as little as about 15, 10, 5 or 2 minutes

In step 207, the wavelength observed of the measured fluorescence emission of step 206 is compared with the wavelength of the fluorescence emission of the conjugated fluorescent nanoparticles that have not been exposed to the antigen. The conjugated fluorescent nanoparticle exhibits a lower emission wavelength upon binding to the antigen.

Such method may be used to detect the presence of the antigen at concentrations equal to or above about 10 cfu/ml or about 4 μg/ml (i.e., the present invention can detect antigen at a concentration of at least about 10 cfu/ml or about 4 μg/ml).

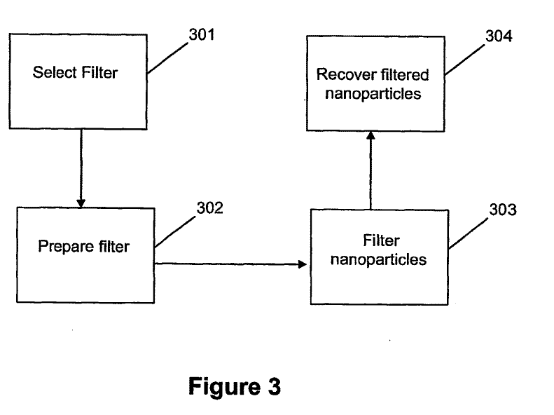

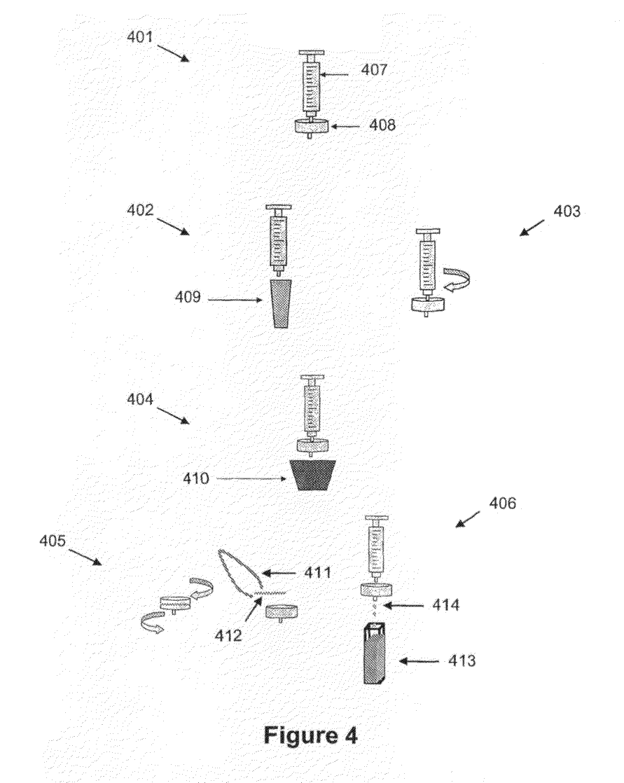

FIGS. 3 and 4 illustrate a disc-filtration method that can be used in an embodiment of the present invention. In step 301, a filtering material (such as a disc), that has a pore size selected dependant upon the size of the antigen, is selected. For instance, the a disc holder (such as a Swinex disc holder), which may also be referred to as a cartridge, is loaded with a filter disc (such as 0.1 μm 0.22 μm/0.45 μm), which is autoclaved prior to loading. Such pore size is selected such that the unbounded conjugated fluorescent nanoparticles will ash through the filter, while the conjugated fluorescent nanoparticles bounded to the antigens will not.

In step 302, the filter can be prepared by washing it with deionized (DI) water or with a prepared buffer. For instance, as shown in 401 of FIG. 4, a syringe 407 (such as a 1 ml syringe) filled with DI water (or a prepared buffer) can be utilized to wash the filter 412 within the disc holder 408.

In step 303, the resulting material, usually incubated, is then passed through the disc holder and filtrate is collected. For instance, as shown in 402 of FIG. 4, the 1 ml syringe 407 can be utilized to draw the resulting material 409 (which also may be referred to as the reacted sample) into the syringe 407. As shown in 403, the prepared disc holder 408 with the filter 412 is attached to the syringe 407.

As shown in 404 of FIG. 4, the syringe 407 is then used to expel the resulting material 409 through the filter 412, by passing the resulting material 409 through the disc holder 408. The filtrate 410, which is the fluid that passes through the disc holder 408 may be deposited in a biohazardous bin. Furthermore, this washing process may be repeated one or more times (using, for example, washing with 1 ml of a PBS buffer, three times). For instance, the disc holder may be washed two times.

By these filtration process, the unbounded conjugated fluorescent nanoparticles are filtered out, i.e., the filtrate 410 contains the unbounded conjugates, while the bounded conjugated fluorescent nanoparticles do not pass through the filter 412.

In step 304, the bounded conjugated fluorescent nanoparticles are prepared for exposure, measurement, and observation, as reflected in FIG. 2, steps 205-207. In an embodiment of step 304 and as illustrated in 405 of FIG. 4, the filter 412 inside the disc holder 408 is reversed using forceps 411. Then, as shown in 406 of FIG. 4, the 1 ml syringe 407 is used to pass buffer through the disc holder 408 and collect the outflow 414 (of the resulting material), such as into a cuvette 413. By such process, the bounded conjugated fluorescent nanoparticles are gathered. Alternative to this reverse flushing technique, the filter itself can be tested without the need to reverse flush. Or, the bounded conjugated fluorescent nanoparticles can be washed out in a buffer (such as a PBS buffer). The cuvette 413 with the outflow 414 is then read in a fluorometer, as described above in steps 205 and 206.

In another embodiment, a centrifuge method is used rather than a syringe-filtration method, such as the syringe-filtration method described above. After incubating the sample with the fluorescent nanoparticle, the fluid containing the antigen is then spun in a centrifuge, such as at 14,000 g. The supernate can then be taken out; generally, this is done from the middle. A portion is added to PBS in a cuvette (such as 2 ml of the supernate and an equal part of PBS). A standard fluorometer can then used to measure the sample, such as similar to as previously described.

### EXAMPLES

The following examples are provided to more fully illustrate some of the embodiments of the present invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### Example 1

These test were performed to simulate testing on building surfaces. In these test, a concrete slab was utilized for testing. The concrete slab was sprayed with 10³, 10⁴, 10⁵ and 10⁶ CFU/ml of Male Specific Coliphage (“MS2”) (approved by the U.S. Department of Defense as a viral stimulant). The slab was let to dry. After the concrete slab had dried, a swab was used to rub on the dry area. The swab was then put in 1 ml of PBS buffer and left in the buffer for 15 minutes.

The swab was then taken out and the sample (i.e, the resulting PBS buffer solution) was tested.

QDs, specifically, Qdot 655 nm made by Quantum Dot Corp. (now Invitrogen), were utilized to make the conjugated fluorescent nanoparticle designed to bind to MS2. The standard process for conjugating Quantum Dot's Qdot 655 nm was used and the antibody was obtained from Tetracore, Inc., Rockville, Md. Different amounts of the conjugate material were interacted with the sample and incubated from 15 minutes and the resulting material was exposed, measured, and observed. These trials were repeated to verify the results. From this Example 1, it appeared that 7 μg/ml yielded superior results for MS2, as compared to other concentrations.

### Example 2

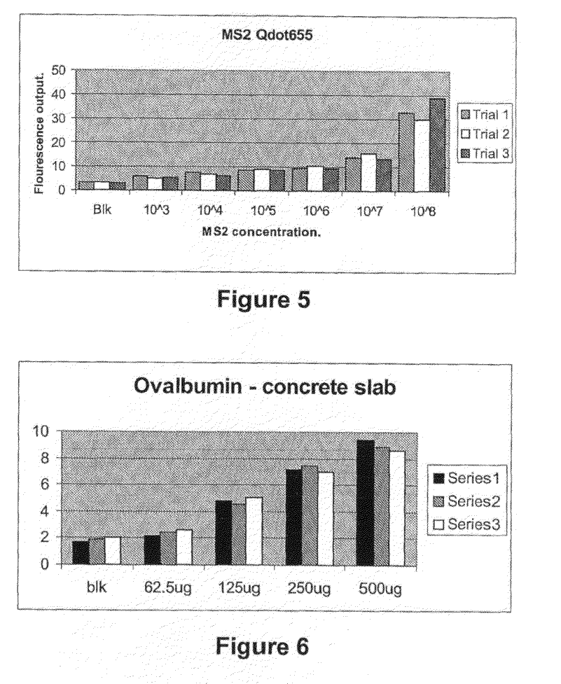

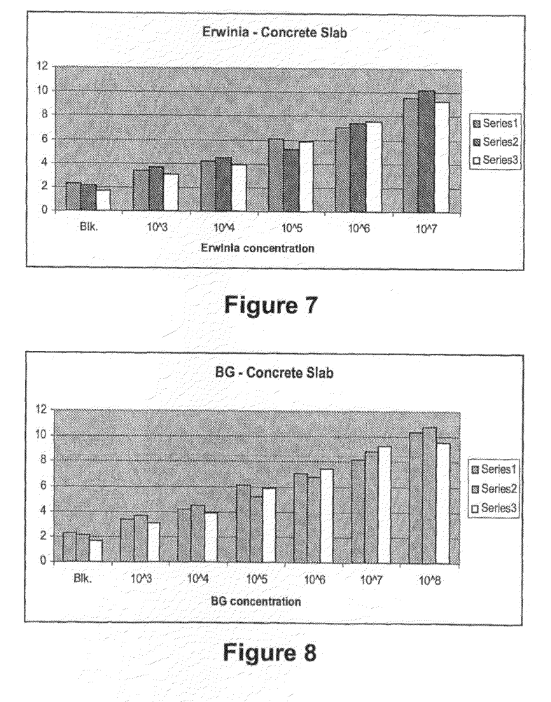

Using 7 μg/ml concentration for the amount conjugate for MS2, the slab surface was sprayed with various concentrations of MS2 (10³, 10⁴, 10⁵, 10⁶, 10⁷, and 10⁸). FIG. 5 reflects the fluorescent output measured for these various concentrations.

### Example 3

The above Example 1 was repeated for Ovalbumin (“OV”) (approved by the U.S. Department of Defense as a Ricin stimulant), except that the concrete slab in Example 1 was sprayed 31.25 ug/ml, 62.5 ug/ml, and 125 ug/ml of OV and the conjugated fluorescent nanoparticle was designed to bind to OV. The antibody was obtained from Sigma, St. Louis, Mo. From Example 3, it appeared that 5 μg/ml yielded superior results for OV, as compared to other concentrations.

### Example 4

The above Example 2 was repeated from OV, except that the concrete slab in Example 1 was sprayed 31.25 ug/ml, 62.5 ug/ml, 125 ug/ml, 250 ug/ml and 500 ug/ml, the conjugated fluorescent nanoparticle was designed to bind to OV, and the conjugate concentration for OV was 5 μg/ml. FIG. 6 reflects the fluorescence output measured for the various concentrations of OV. The fluorescence output was lower as compared with MS2, which is believed to be due to the size of the OV protein.

### Example 5

The above Example 1 was repeated for Erwinia herbicola (EH) (approved by the U.S. Department of Defense as a Yersinia pestis stimulant), except that the concrete slab in Example 1 and the conjugated fluorescent nanoparticle was designed to bind to EH. The antibody was obtained from Morphosys USA, Brentwood, N.H. Furthermore, the incubation time required about 20 minutes. From Example 5, it appeared that 6 μg/ml yielded superior results for EH, as compared to other concentrations.

### Example 6

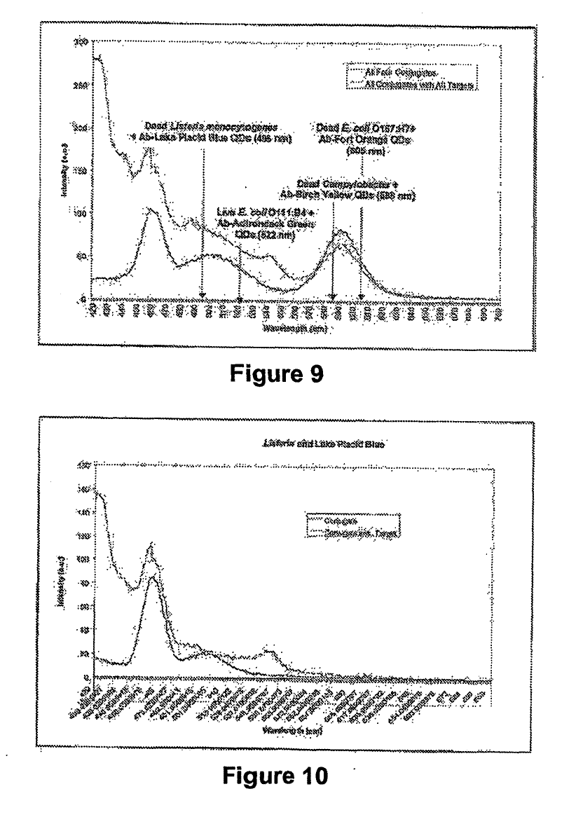

The above Example 2 was repeated from EH, except that the concrete slab in Example 1 was sprayed at 10³, 10⁴, 10⁵, 10⁶, and 10⁷ concentrations, the conjugated fluorescent nanoparticle was designed to bind to EH, and the conjugate concentration for EH was 6 μg/ml. FIG. 9 reflects the fluorescence output measured for the various concentrations of EH.

### Example 7

The above Example 1 was repeated for Bacillus Globigii (BG) (approved by the U.S. Department of Defense as an anthrax stimulant), except that the concrete slab in Example 1 the conjugated fluorescent nanoparticle was designed to bind to BG. The antibody was obtained from Tetracore, Inc., Rockville, Md. From Example 7, it appeared that 7 μg/ml yielded superior results for BG, as compared to other concentrations.

### Example 8

The above Example 2 was repeated from BG, except that the concrete slab in Example 1 was sprayed at 10³, 10⁴, 10⁵, 10⁶, 10⁷, and 10⁸ concentrations, the conjugated fluorescent nanoparticle was designed to bind to BG, and the conjugate concentration for BG was 7 μg/ml. FIG. 8 reflects the fluorescence output measured for the various concentrations of BG.

Examples 1-8 reflect that each of MS2, OV, EH, and BG were detected utilizing the present invention.

### Example 9

The present invention can also be used to detect a mixture or “cocktail” of various bioterrorism assays, and can be used to discriminate some or all of the components spectrally. In this Example, four different bacterial immuno-QD assays were mixed together. Specifically, these four bacterial immuno-QD assays were (a) dead Listeria monocytogenes, (b) live E. coli O111:B4, (c) dead Campylobacter, and (d) dead E. coli O157:H7. Four different conjugated fluorescent nanoparticle were designed, with each designed to bind to one of these four bacterial immuno-QD assays. Respectively, these were (a) Ab-Lake Placid Blue QDs (498 nm), (b) Ab-Adirondack Green QDs (522 nm), (c) Ab-Birch Yellow QDs (588 nm), and (d) Ab-Fort Orange Qds (605 nm). In this example, Evitag QDs (of Evident Technology, Troy, N.Y.) were utilized.

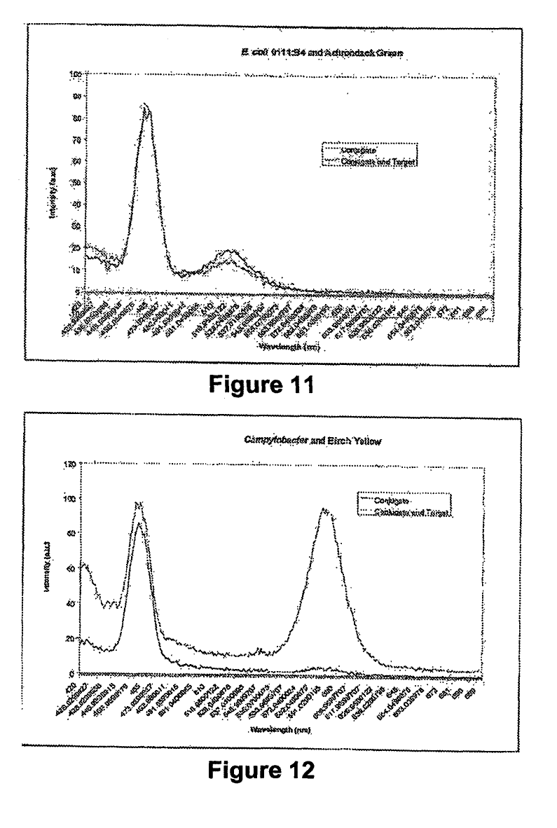

FIG. 9 reflects the unfiltered spectra that was received from this mixture. FIGS. 10-12 illustrate the spectra from three of these bacterial immuno-QD assays, namely (a) dead Listeria monocytogenes and Ab-Lake Placid Blue QDs (498 nm), (b) live E. coli O111:B4 and Ab-Adirondack Green QDs (522 nm), and (c) dead Campylobacter and Ab-Birch Yellow QDs (588 nm), respectively. Software algorithms, like those devised and published by the Naval Research Laboratory for discrimination of four different biotoxin-antibody-QD assays can be used to further identify the suspected antigens. [See, e.g., Goldman, E. R, et al., “Multiplexed Toxin Analysis Using Four Colors of Quantum Dot Fluororeagents, Anal. Chem., 76(3) (2004), pp. 684-88].

### Example 10

Food samples (dry soup mix) were prepared with concentrations of 10¹, 10², 10³, and 10⁴ cfu/ml of E. coli O157:H7 bacteria The food sample containing the concentration of 10¹cfu/ml of E. coli O157:H7 bacteria was prepared as follows. 25 ml of buffer was added to 3 g of the food sample to form a mixture. 10¹ cfu/ml of E. coli O157:H7 bacteria was spiked into this mixture and then mixed thoroughly. 1 ml was taken of this composition and put into a first tube. This was then centrifuged at 14,000 rpm for 5 minutes. The supernatant was collected and placed in a second tube. The first tube with pellet was discarded. The other food samples with concentrations of 10², 10³, and 10⁴ cfu/ml of E. coli O157:H7 bacteria were prepared by a similar process.

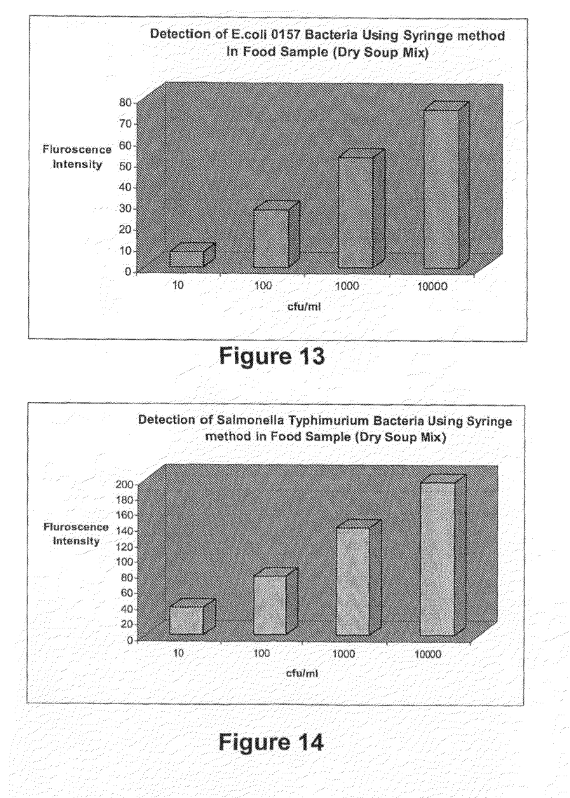

These various food samples were tested using an embodiment of the present invention. The collected supernatant in each of the various tubes were incubated in a solution comprising conjugated fluorescent nanoparticles designed to bind to E. coli O157:H7 bacteria. The incubated supernatant were then subjected to the syringe-filtration method. The fluorescence output for the various food samples was then measured. FIG. 13 reflects the fluorescence output measured for the various concentrations of E. coli O157:H7 bacteria Example 10 reflects that E. coli O157:H7 bacteria was detected utilizing the present invention, even for concentrations as low as 10 cfu/ml.

### Example 11

Example 10 was repeated, except that the food samples were prepared with concentrations of Salmonella typhimurium bacteria and the conjugated fluorescent nanoparticles were designed to bind to Salmonella typhimurium bacteria. FIG. 14 reflects the fluorescence output measured for the various concentrations of Salmonella typhimurium bacteria. Example 10 reflects that Salmonella typhimurium bacteria was detected utilizing the present invention, even for concentrations as low as 10 cfu/ml.

### Example 12

Example 10 was repeated, except that a serum sample was utilized in lieu of food samples, the serum samples were spiked with concentrations of 4 μg, 8 μg, 16 μg, 32 μg, and 64 g of OV, and the conjugated fluorescent nanoparticles were designed to bind to OV. The serum sample containing the 4 μg concentration of OV was prepared as follows. 2 ml of blood was drawn and centrifuged to separate the serum out. Thereafter 500 μl of the serum was spiked with 4 ug/ml of OV. PBS was then added to yield a 1 ml serum sample. The other to serum samples with concentrations of 8 μg, 16 μg, 32 μg, and 64 μg of OV were prepared by a similar process.

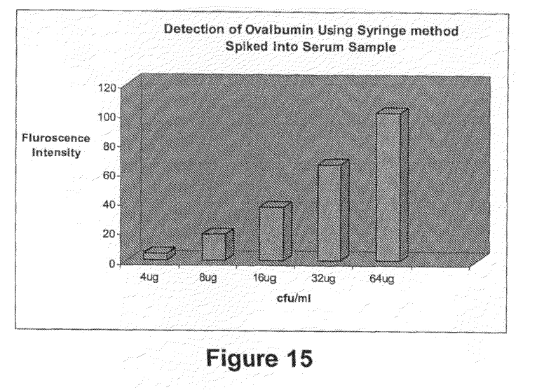

Similar to Examples 11 and 12, the serum samples were incubated with the conjugated fluorescent nanoparticles were designed to bind to OV, subjected to the syringe-filtration method, and the fluorescence output for the various serum samples was then measured. FIG. 15 reflects the fluorescence output measured for the various concentrations of OV. Example 12 reflects that OV was detected utilizing the present invention, even for concentrations as low as 4 μg. Moreover, this Example 12 reflects that the present invention may be used to detect small sized antigens. Certain markers, such as cardiac markers, are small proteins, and thus may be detectable by the present invention.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above and below described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

It is therefore, contemplated that the claims will cover any such modifications or embodiments that fall within the true scope of the invention.

1. A method of detecting an antigen comprising:
(a) obtaining a sample from a surface or source where an antigen is suspected to be;
(b) obtaining a fluorescent nanoparticle conjugated to a substance capable of binding specifically to the antigen to form a bounded conjugated fluorescent nanoparticle;
(c) interacting the sample with the fluorescent nanoparticle to form a resulting material, wherein, if the antigen are present in the sample, the resulting material comprises conjugated fluorescent nanoparticles bound to the antigen;
(d) exposing the conjugated fluorescent nanoparticles of the resulting material to a wavelength of light capable of exciting the conjugated fluorescent nanoparticle;
(e) measuring fluorescence emission of the conjugated fluorescent nanoparticle; and
(f) observing the wavelength of the measured fluorescence emission of said step of measuring in comparison with the wavelength of the fluorescence emission of the conjugated fluorescent nanoparticles that have not been exposed to the antigen, wherein the conjugated fluorescent nanoparticle exhibits a lower emission wavelength upon binding to the antigen.

2. The method of claim 1 furthering comprising filtering the resulting material before said step of exposing the resulting material.

3. The method of claim 2, wherein the filtering step comprises a process selected from the group consisting of syringe-filtration methods, centrifuge methods, and combinations thereof.

4-5. (canceled)

6. The method of claim 2, wherein in said filtering step, a filter is used that has a pore size operable for such that conjugated fluorescent nanoparticle bound to antigen of the first type of antigen cannot pass through the filter while conjugated fluorescent nanoparticle not bound to antigen of the first type of antigen can pass through the filter.

7. The method of claim 6, wherein the filtration step comprises a reverse flushing technique to collect the bound conjugated fluorescent nanoparticle in the resulting material.

8-10. (canceled)

11. The method of claim 1, wherein the step of obtaining a sample comprises a swab-test.

12. The method of claim 1, further comprising incubating the resulting material before said step of exposing the resulting material.

13. The method of claim 12, wherein the said step of incubating is performed for at least about 10 minutes.

14. (canceled)

15. The method of claim 1, wherein the antigen is selected from the group consisting of a bacteria, a virus, and a small protein.

16-17. (canceled)

18. The method of claim 1, wherein the antigen is selected from the group consisting of viral particles, ricin, yersinia pestis, and anthrax.

19. The method of claim 1, wherein the substance capable of binding specifically to the antigen is an antibody.

20. The method of claim 1, wherein the antigen is a bacteria and the substance capable of binding specifically to the bacteria is an aptamer.

21. The method of claim 1, wherein the fluorescent nanoparticle comprises cadmium selenide/zinc sulfate.

22. The method of claim 1, wherein the fluorescent nanoparticle comprises a quantum confined nanosize particle.

23. The method of claim 22, wherein the fluorescent nanoparticle is a metal oxide with a lanthanide core.

24-28. (canceled)

29. The method of claim 1, wherein the method of detecting the antigen detects the presence of the antigen at a concentration of at least about 10 cfu/ml.

30-31. (canceled)

32. The method of claim 1, wherein the method of detecting the antigen detects the presence of the antigen at a concentration of at least about 4 μg/ml.

33. (canceled)

34. The method of claim 1, wherein
(i) a different antigen is further suspected to be with the surface or the source, wherein the different antigen is different than the antigen;
(ii) a different fluorescent nanoparticle is obtained that is conjugated to a substance capable of binding specifically to the different antigen;
(iii) if different antigen are present in the sample, the resulting material comprises different conjugated fluorescent nanoparticles bound to the different antigen;
(iv) exposing the different conjugated fluorescent nanoparticle of the resulting material to a wavelength of light capable of exciting the different conjugated fluorescent nanoparticle;
(v) measuring fluorescence emission of the different conjugated fluorescent nanoparticle; and
(vi) observing the wavelength of the measured fluorescence emission of said step of measuring fluorescence emission of the different conjugated fluorescent nanoparticle in comparison with the wavelength of the fluorescence emission of the different conjugated fluorescent nanoparticles that have not been exposed to the different antigen, wherein the different conjugated fluorescent nanoparticle exhibits a lower emission wavelength upon binding to the different antigen.

35. The method of claim 1, wherein the surface or the source is suspected to contain said antigen that has been used as a biological warfare agent, and said method is utilized to detect the biological warfare agent.

36-37. (canceled)

38. The method of claim 1, wherein the sample was obtained from a body fluid.

39. The method of claim 38, wherein the body fluid was selected from the group consisting of blood, urine, stool sample, saliva, and spinal fluid.

40. (canceled)

41. The method of claim 1, wherein the method is utilized to detect a disease in a human or an animal.

42. The method of claim 1, wherein the method detects a disease in a human or an animal.

43. (canceled)

44. The method of claim 1, wherein said steps (e) and (f) occur collectively in at most about 15 minutes.

45-48. (canceled)

49. The method of claim 1, wherein a fluorometer is utilized during step (e).

50. (canceled)
